(19) United States
(12) Patent Application Publication
(10) Pub. No.: US 2018/0085102 A1
(43) Pub. Date: Mar. 29, 2018

(54) MEDICAL DEVICE

(71) Applicant: OLYMPUS CORPORATION, Tokyo (JP)

(72) Inventor: Chisato KIKUCHI, Tokyo (JP)

(73) Assignee: OLYMPUS CORPORATION, Tokyo (JP)

(21) Appl. No.: 15/827,484

(22) Filed: Nov. 30, 2017

Related U.S. Application Data
(63) Continuation of application No. PCT/JP2016/066644, filed on Jun. 3, 2016.

(51) Int. Cl. A61B 17/00; A61B 90/00; A61B 17/04

(52) U.S. Cl. CPC A61B 17/0057; A61B 90/08; A61B 17/0482; A61B 2090/062; A61B 2090/0807; A61B 17/00234; A61B 2017/00955; A61B 2017/00907; A61B 2090/3945; A61B 2017/00929; A61B 2017/00951; A61B 2017/0065; A61B 2017/0061; A61B 2017/00561

(57) ABSTRACT

A medical device including: an insertion portion configured to be inserted into a body; and a sheet provided at a distal end of the insertion portion, the sheet being configured to cover a body wall. The sheet including: an attaching portion provided around a periphery of the sheet, the attaching portion being configured to directly contact with the body wall; and a notifying portion for notifying a surgeon that an incision has been made through the body wall and towards a surface of the sheet.

### CROSS-REFERENCE TO RELATED APPLICATIONS

This application is a continuation of International Application PCT/JP2016/066644, with an international filing date of Jun. 3, 2016, which is hereby incorporated by reference herein in its entirety.

### BACKGROUND

### Field

The present disclosure relates to a medical device.

### Prior Art

When treating the lining of the digestive tract, it is required to prevent the exterior of the digestive tract from being contaminated by the contents of the digestive tract. Therefore, methods for treating the lining while keeping the interior and the exterior of the digestive tract isolated from each other have been proposed (for example, see PCT Publication No. 2009-540963 and University of Tokyo Gastroenterology Digestive Tract Group, “NEWS (Non-exposed Endoscopic Wall-inversion Surgery)”, [online], [accessed on Mar. 9, 2016], Internet <URL: http://www.todai-shoukakan.com/cont3/13.html).

The equipment described in PCT Publication No. 2009-540963 is provided with a capping member that expands like a sheet inside the digestive tract, thus blocking an opening formed in the digestive-tract wall. With the surgical method proposed in “NEWS (Non-exposed Endoscopic Wall-inversion Surgery)”, in order to remove a tumor formed in the gastric mucosa without opening up the interior of the stomach, only the outer portion of the stomach wall is excised from outside the stomach, the excision site is subsequently sutured, and then, the remaining portion of the stomach wall is excised from inside the stomach.

### SUMMARY

Accordingly, a medical device is provided. The medical device comprising: an insertion portion configured to be inserted into a body; and a sheet provided at a distal end of the insertion portion, the sheet being configured to cover a body wall, the sheet comprising: an attaching portion provided around a periphery of the sheet, the attaching portion being configured to directly contact with the body wall; and a notifying portion for notifying a surgeon that an incision has been made through the body wall and towards a surface of the sheet.

### BRIEF DESCRIPTION OF DRAWINGS

FIG. 1A is a configuration diagram of a medical device according to an embodiment in an open form.

FIG. 1B is a front view of the medical device in FIG. 1A viewed from a distal-end side.

FIG. 2 is a configuration diagram of the medical device in FIG. 1A in a closed form.

FIG. 3 is a diagram for explaining the manner in which the medical device in FIG. 1A is used.

FIG. 4 is a diagram for explaining the manner in which the medical device in FIG. 1A is used.

FIG. 5A is a configuration diagram of a modification of the medical device in FIG. 1A.

FIG. 5B is a front view of the medical device in FIG. 5A viewed from a distal-end side.

FIG. 6 is a configuration diagram of another modification of the medical device in FIG. 1A.

FIG. 7 is a configuration diagram of another modification of the medical device in FIG. 1A.

FIG. 8 is a configuration diagram of another modification of the medical device in FIG. 1A.

FIG. 9 is a configuration diagram of another modification of the medical device in FIG. 1A.

### DETAILED DESCRIPTION

A medical device 1 according to an embodiment will be described below with reference to the drawings.

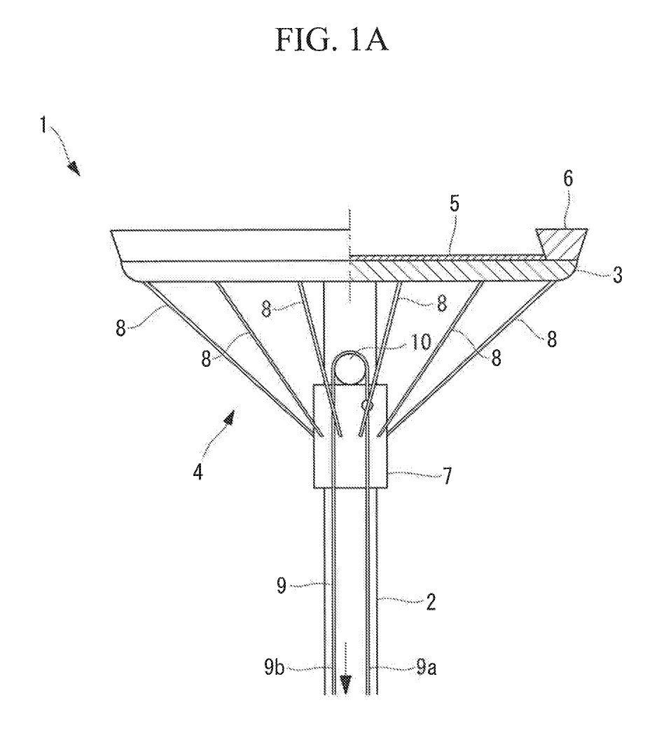

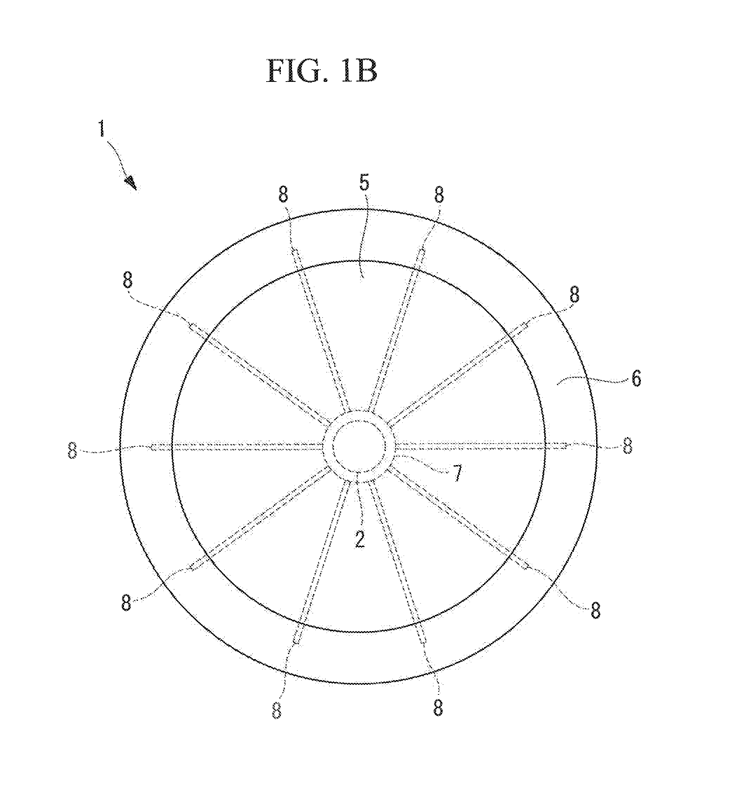

As shown in FIGS. 1A and 1B, the medical device 1 according to this embodiment is provided with: a long, thin elongated insertion portion 2; a sheet portion 3 (alternatively referred to as a “sheet”) that is provided at a distal end of the elongated insertion portion 2 and that can be expanded and contracted in the radial direction with respect to the elongated insertion portion 2; a opening/closing mechanism 4 that causes the sheet portion 3 to be deformed; and a notifying portion 5 and an attaching portion 6 that are provided on the sheet portion 3.

The medical device 1 is used by being inserted into a channel of a lumen endoscope. The lumen endoscope is a small-diameter, flexible endoscope to be used in a lumen such as the stomach or the intestine.

The elongated insertion portion 2 has a diameter that allows insertion thereof into the channel of the lumen endoscope. In addition, the elongated insertion portion 2 has flexibility that allows bending thereof in conformity to bending of the lumen endoscope in the body.

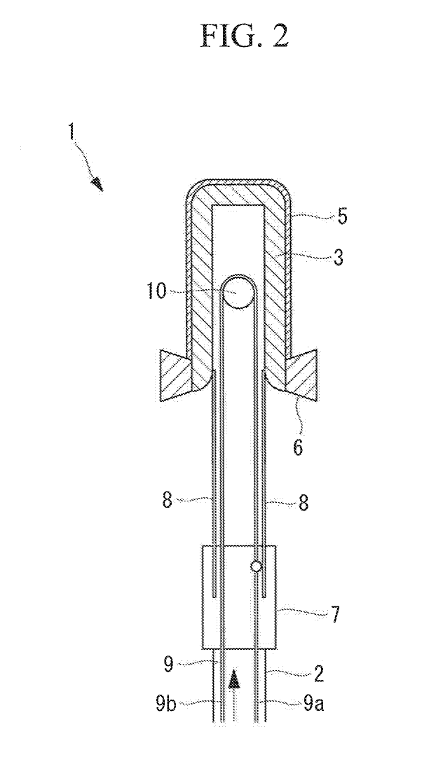

The sheet portion 3 is formed of a circular sheet-like member that is formed of a resin material having flexibility, such as silicone rubber, urethane rubber, or vinyl chloride. The sheet portion 3 has a distal-end surface and a proximal-end surface that face each other in the thickness direction, and is joined with the distal end of the elongated insertion portion 2 at substantially the center of the proximal-end surface. Such a sheet portion 3 can be deformed like the canopy of an umbrella between the open form shown in FIG. 1A and the closed form shown in FIG. 2. The open form is a form in which the sheet portion 3 is expanded to be flat in a direction that intersects the longitudinal direction of the elongated insertion portion 2. The closed form is a form in which the sheet portion 3 is folded so as to be placed along the longitudinal direction of the elongated insertion portion 2.

The opening/closing mechanism 4 is provided with: a sliding portion 7 that is provided in the elongated insertion portion 2 so as to be movable along the longitudinal direction of the elongated insertion portion 2; a plurality of ribs 8, each of which have an end thereof connected to the proximal-end surface of the sheet portion 3 and an end thereof connected to the sliding portion 7; and a wire 9 that drives the sliding portion 7 along the longitudinal direction of the elongated insertion portion 2.

The plurality of ribs 8 are arrayed in a radiating manner centered on the elongated insertion portion 2 with spaces therebetween in the circumferential direction of the elongated insertion portion 2. The distal ends of the individual ribs 8 are secured to the periphery of the proximal-end surface of the sheet portion 3.

The elongated insertion portion 2 is provided with a shaft portion 10 that extends in the radial direction of the elongated insertion portion 2 at a position that is closer to the distal end than the sliding portion 7 is. The wire 9 is threaded around the shaft portion 10, and two end portions 9a and 9b of the wire 9 are disposed at the proximal end of the elongated insertion portion 2. By doing so, the two end portions 9a and 9b of the wire 9 are configured so as to be moved in mutually opposing directions. In addition, an intermediate position of the wire 9 is secured to the sliding portion 7. Therefore, by pulling the one end portion 9a of the wire 9 toward the proximal end, the sliding portion 7 is moved toward the proximal end, and thus, the ribs 8 pull the sheet portion 3 into the closed form. On the other hand, by pulling the other end portion 9b of the wire 9 toward the proximal end, the sliding portion 7 is moved toward the distal end, and thus, the ribs 8 push and expand the sheet portion 3 into the open form. In order to assist the deformation of the sheet portion 3 from the closed form to the open form, the ribs 8 may possess an elastic force that biases the sheet portion 3 toward the distal end. For example, the ribs 8 may be formed of elastic members such as springs.

The notifying portion 5 is formed of a color layer (hereinafter also referred to as color layer 5) provided on the distal-end surface of the sheet portion 3. The color layer 5 covers the entire distal-end surface of the sheet portion 3, excluding the attaching portion 6. On the surface of the color layer 5, water repellent treatment may be applied in order to prevent attachment of a liquid such as blood or the like.

The color layer 5 can have a color that is different from that of the body wall so as to be conspicuous in the body, and have a color that is complementary to the colors of the body wall and blood or a color close to a complementary color. Furthermore, the color of the color layer 5 can be a color with which an afterimage is unlikely to be generated. Examples of such color include green and blue. Although the color of the color layer 5 may be uniform over the entirety thereof, a pattern (for example, a stripe pattern or a checkered pattern) formed of a plurality of colors may be applied to the color layer 5 in order to further increase the visibility of the color layer 5 in the body.

The attaching portion 6 is formed of a biological adhesive, such as a fibrin glue, that is applied to the distal-end surface of the sheet portion 3. The biological adhesive is applied, in an annular manner, around the entire circumference of the periphery of the surface of the sheet portion 3, and protrudes farther than the distal-end surface of the sheet portion 3 in the thickness direction of the sheet portion 3. The amount by which the attaching portion 6 protrudes from the distal-end surface of the sheet portion 3 is designed so that the outer size of the attaching portion 6 in the radial direction of the elongated insertion portion 2 in the closed form of the sheet portion 3 becomes smaller than the inner size of the channel of a lumen endoscope 30. The biological adhesive is a thermoresponsive adhesive that exhibits adhesiveness by being heated to the body temperature. Therefore, the sheet portion 3 is secured to the body wall by means of the adhesiveness that the attaching portion 6 exhibits by being in contact with the body wall.

Next, the operation of the thus-configured medical device 1 will be described.

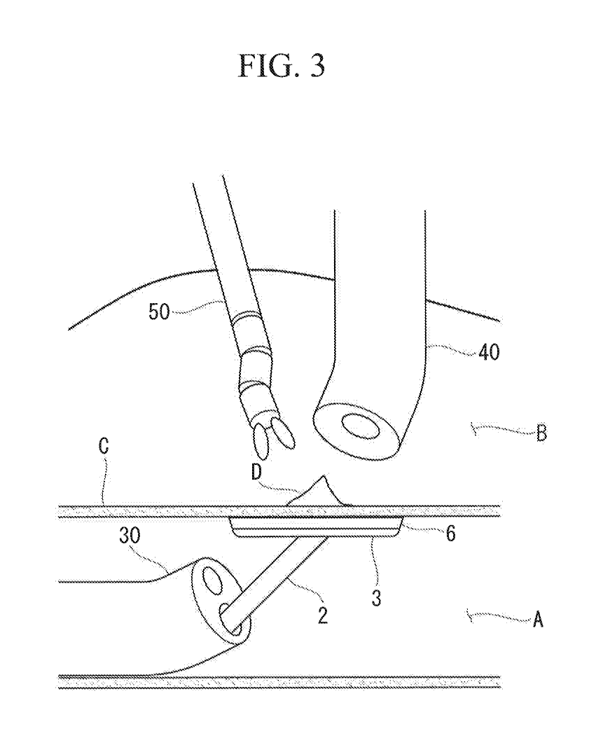

As shown in FIG. 3, the medical device 1 according to this embodiment is used in a surgery for excising, by using the lumen endoscope 30 inserted into a lumen A and a body cavity endoscope 40 inserted into a body cavity B, an affected portion D, such as a tumor, formed in a wall C of the lumen A that separates the lumen A and the body cavity B. The lumen A is, for example, the stomach or the intestine, and the body cavity B is, for example, the abdominal cavity.

A surgeon sets the sheet portion 3 to the closed form by pulling the one end portion 9a of the wire 9, which extends from the proximal end of the elongated insertion portion 2, toward the proximal end, and places the sheet portion 3 in the vicinity of the affected portion D via the channel of the lumen endoscope 30 inserted into the lumen A. Next, the surgeon expands the sheet portion 3 into the open form by pulling the other end portion 9b of the wire 9.

Next, while observing the affected portion D by using the lumen endoscope 30 from inside the lumen A, the surgeon places the sheet portion 3 facing the surface of the wall C so that the entire affected portion D is covered by the sheet portion 3, thus bringing the attaching portion 6 into close contact with the wall C. The biological adhesive of the attaching portion 6 heated by the body temperature exhibits adhesiveness, thus securing the sheet portion 3 to the wall C. At this time, the space between the wall C and the sheet portion 3 is sealed by the attaching portion 6 that is provided around the entire circumference of the periphery of the sheet portion 3, and thus, the affected portion D is isolated from the internal space in the lumen A.

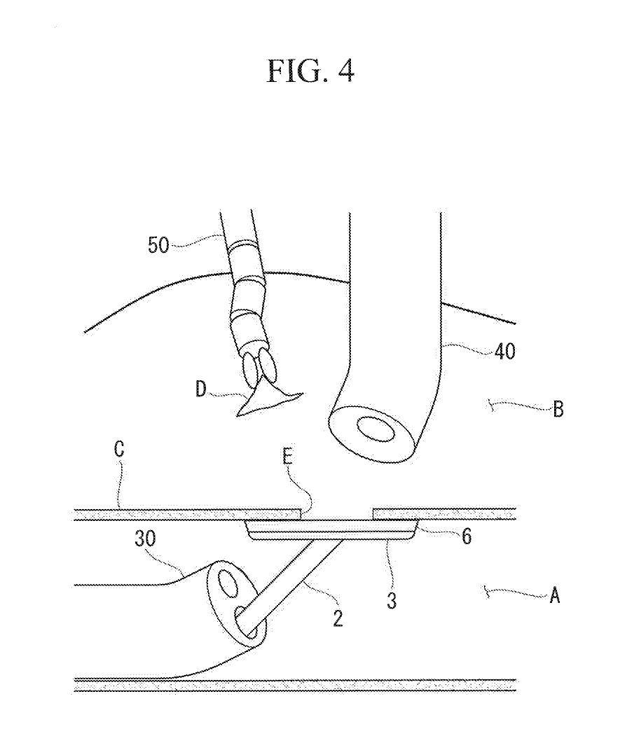

Next, as shown in FIG. 4, while observing the affected portion D from outside the lumen A by using the body cavity endoscope 40 inserted into the body cavity B, the affected portion D is excised from the wall C by making an incision in the area surrounding the affected portion D by using a treatment tool 50 (for example, a scalpel, a high-frequency knife, an ultrasonic knife or scissors) inserted into the body cavity B. Because a hole E formed in the wall C due to excision of the affected portion D is blocked by the sheet portion 3 and the attaching portion 6, the lumen A does not communicate with the body cavity B via the hole E. After excising the affected portion D, the surgeon completes treatment of the wall C by suturing the hole E.

In this case, with this embodiment, when the incision is made in the wall C from the body cavity B into the lumen A, the color layer 5 that covers the wall C on the lumen A is exposed to the body cavity B via the incision site, and thus, the color layer 5 is observed in the incision site in the image captured by the body cavity endoscope 40. When the color layer 5, which has a conspicuous color with respect to the wall C, appears in the image in this way, the surgeon receives a visual notification that the wall C has been penetrated into the lumen A. Therefore, the surgeon can quickly and easily recognize penetration of the wall C, and thus, it is possible to appropriately manipulate the treatment tool 50 so that the sheet portion 3 is not damaged by the treatment tool 50. By doing so, there is an advantage in that, while treating the wall C, it is possible to reliably keep the lumen A and the body cavity B isolated from each other by means of the sheet portion 3 and the attaching portion 6 that block the incision site from the lumen A. Furthermore, because excision of the affected portion D may be performed only from the body cavity B, there is an advantage in that it is possible to reduce the amount of time required to perform treatment.

In this embodiment, the sheet portion 3 can have a thickness of 2 mm or greater. With the sheet portion 3 having a thickness of 2 mm or greater, even if the treatment tool 50 comes into contact with the sheet portion 3, it is possible to more reliably prevent the sheet portion 3 from being penetrated by the treatment tool 50.

In this embodiment, although the attaching portion 6 is formed of the thermoresponsive adhesive, alternatively, the attaching portion 6 may be formed of a moisture-reactive biological adhesive that exhibits adhesiveness via a reaction with water. By doing so also, it is possible to secure the sheet portion 3 to the wall C by causing the attaching portion 6 to exhibit adhesiveness via contact with the wall C.

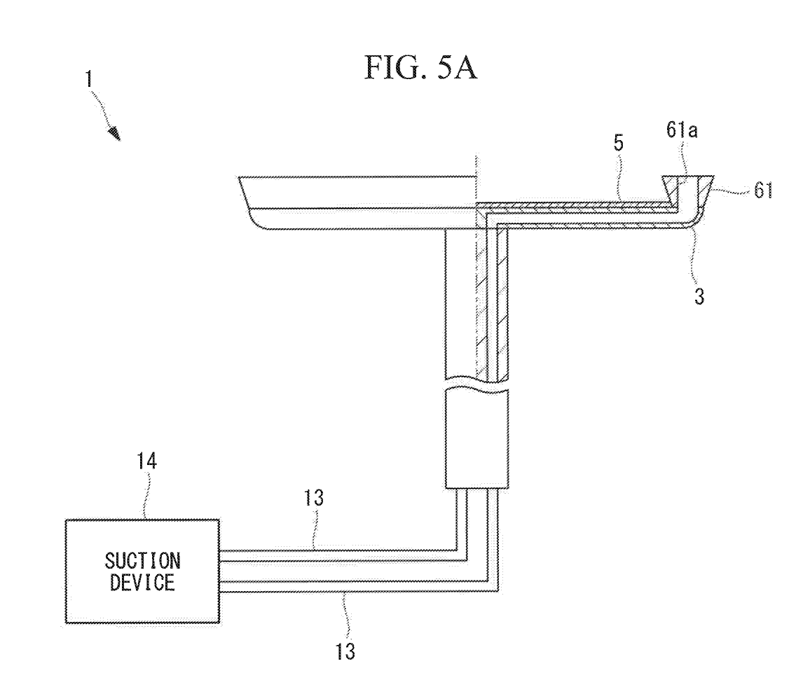

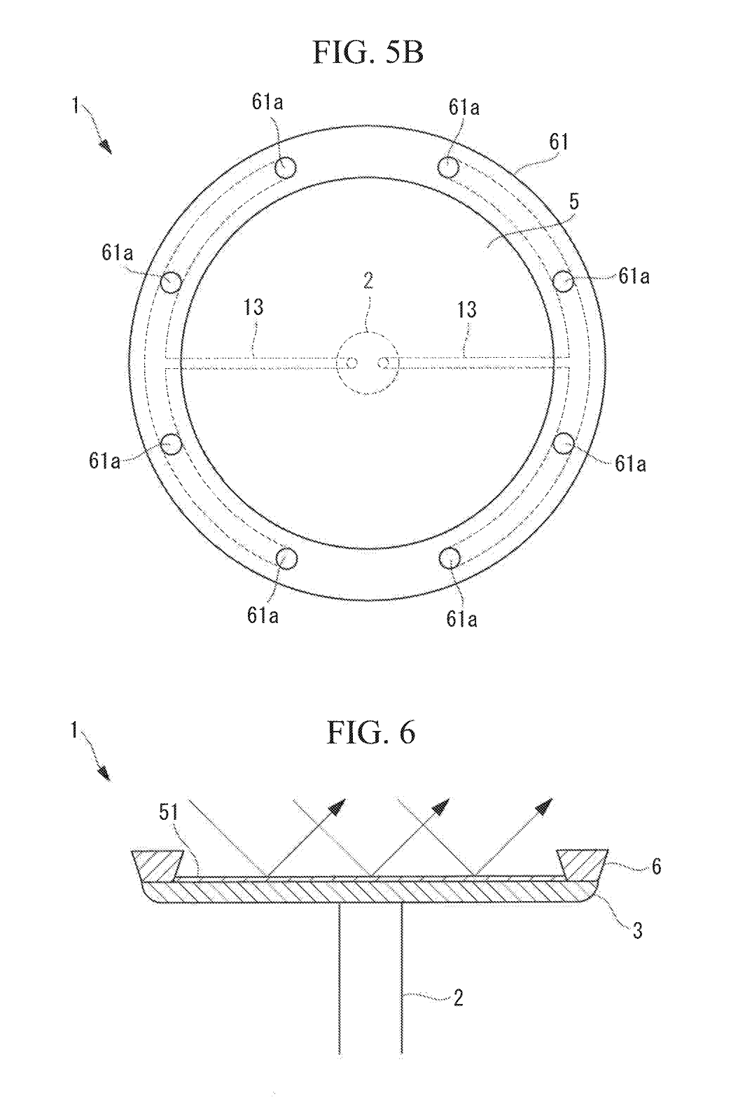

Alternatively, as shown in FIGS. 5A and 5B, an attaching portion 61 may be configured to be secured to the wall C by means of adhesion. The attaching portion 61 is provided around the entire circumference of the periphery of the distal-end surface of the sheet portion 3, and is formed of an annular member that protrudes from the distal-end surface of the sheet portion 3. As shown in FIG. 5B, a plurality of suction ports 61a, which are arranged with spacings therebetween in the circumferential direction, are provided at the distal-end surface of the attaching portion 61.

The plurality of suction ports 61a are connected, via suction pipes 13 that pass through the interior of the elongated insertion portion 2, to a suction device 14 (for example, a pump or a syringe) that is disposed at the proximal end of the elongated insertion portion 2 and that generates a suction force. The distal-end surface of the attaching portion 6 adheres to the wall C due to the suction force generated by the suction device 14. Although FIGS. 5A and 5B show the plurality of suction ports 61a that are arranged with spacings therebetween in the circumferential direction, the suction ports may have a circular shape or an arc-like shape that extends around the circumferential direction of the attaching portion 61.

Note that, in FIGS. 5A to 9, the illustration of the opening/closing mechanism 4 is omitted in order to simplify the drawings.

In this embodiment, although the notifying portion is formed of the color layer 5 that has a color that is different from that of the wall C, alternatively, as shown in FIG. 6, the notifying portion may be formed of a reflector 51 that covers the entire distal-end surface of the sheet portion 3 and that reflects light. Illumination light that is radiated onto the wall C from the distal-end surface of the body cavity endoscope 40 is reflected toward the body cavity B by the reflector 51 exposed from the incision site, and the reflected light is observed in the image captured by the body cavity endoscope 40. By doing so, it is possible to visually notify the surgeon about penetration of the wall C.

The reflector 51 may be a metal film that is formed by coating the distal-end surface of the sheet portion 3, or may be a metal foil attached to the distal-end surface of the sheet portion 3. Alternatively, the reflector 51 may be a metal sheet having a mirror-finish surface. The surface of the reflector 51 may be an irregular surface oriented in various directions so as to reflect light in various directions. In addition, the reflector 51 may have a pattern (for example, a stripe pattern or a checkered pattern) formed of reflective regions having high optical reflectance and non-reflective regions having low optical reflectance.

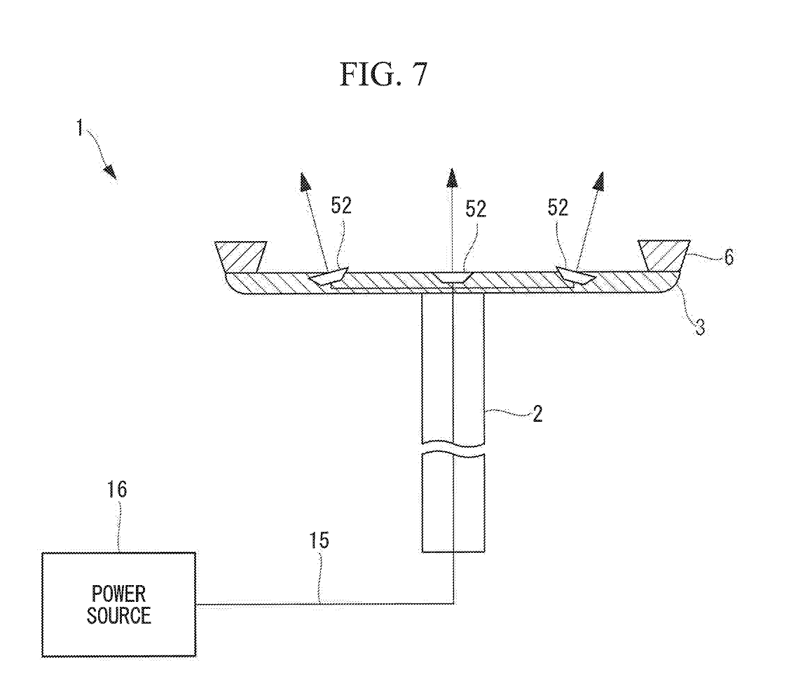

As shown in FIG. 7, the notifying portion may be formed of light-emitting bodies 52 that are provided at the distal-end surface of the sheet portion 3 and that emit light. By doing so also, light emitted into the body cavity B from the light-emitting bodies 52 exposed from the incision site is observed in the image, and thus, it is possible to visually notify the surgeon about penetration of the wall C.

The light-emitting bodies 52 are constituted of, for example, a plurality of LEDs or organic EL devices that are arrayed at the distal-end surface of the sheet portion 3. The light-emitting bodies 52 are connected to a power source 16 that is disposed at the proximal end of the elongated insertion portion 2 via an electric wire 15 that passes through the interior of the elongated insertion portion 2 so that power is supplied from the power source 16 via the electric wire 15. The plurality of light-emitting bodies 52 can be disposed so as to emit light in various directions so that light coming from the light-emitting bodies 52 can be recognized regardless of the direction from which the sheet portion 3 is observed. The color of the light emitted by the light-emitting bodies 52 can be, as with the color of the color layer 5, a color that is complementary to the colors of the wall C and blood or a color that is close to a complementary color, and with which an afterimage is unlikely to be generated. The light may flicker or the color of the light may be changed among multiple colors so that the surgeon can more easily recognize the light coming from the light-emitting bodies 52.

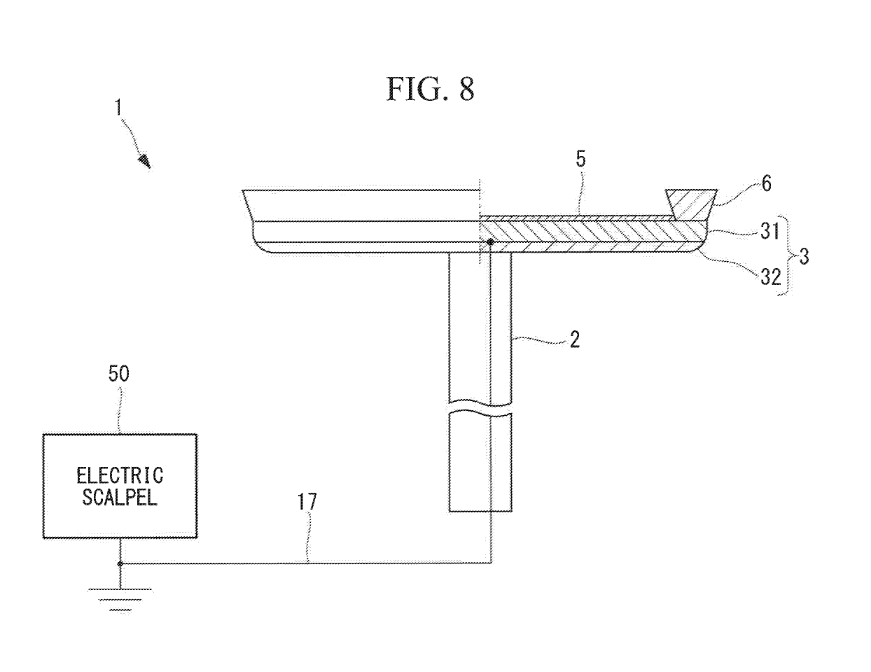

In this embodiment, although the sheet portion 3 is constituted of a single layer formed of a resin material, alternatively, as shown in FIG. 8, the sheet portion 3 may include a conductive layer 31 possessing conductivity and an insulating layer 32 that covers the proximal-end surface of the conductive layer 31 and that has electrical insulating properties. The conductive layer 31 and the insulating layer 32 can be formed over the entire region of the sheet portion 3 in the surface direction of the sheet portion 3.

The conductive layer 31 is connected to a ground of the electric scalpel 50 via ground line 17 that passes through the interior of the elongated insertion portion 2. Because a current flows from the electric scalpel 50 to the ground via the conductive layer 31 and the ground line 17 when the electric scalpel 50 penetrates the notifying portion 5 and comes into contact with the conductive layer 31, an incision is not made in the conductive layer 31 by the electric scalpel 50. By doing so, it is possible to more reliably prevent the sheet portion 3 from being damaged.

Furthermore, because an incision is also not made in the insulating layer 32 by the electric scalpel 50, it is possible to more reliably prevent the sheet portion 3 from being damaged.

In this embodiment, although the surgeon is visually made to recognize, by means of an image, the notifying portion 5 that is exposed to the body cavity B via the incision site, whereby the surgeon is notified about penetration of the wall C, alternatively, penetration of the wall C may be notified by using other means.

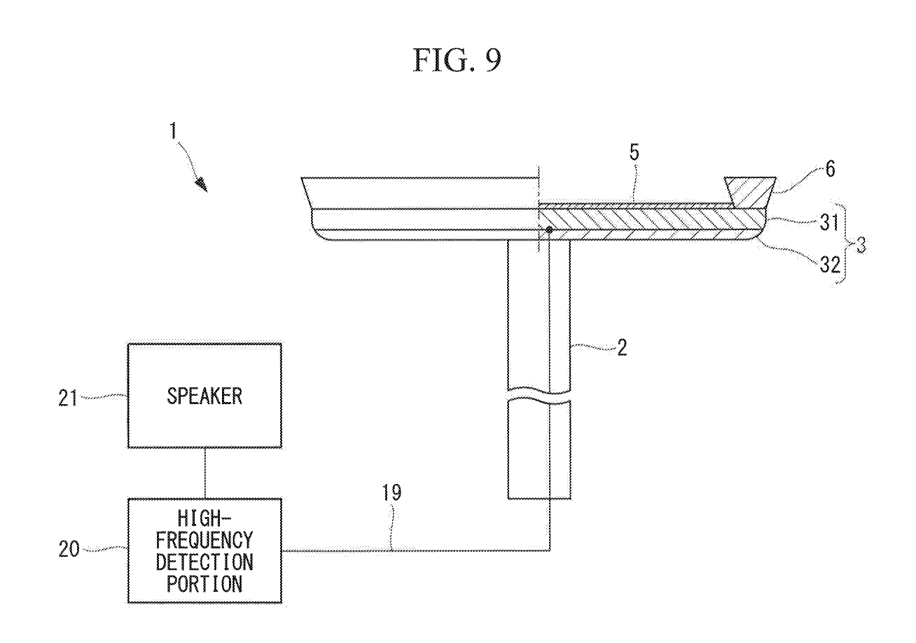

For example, as shown in FIG. 9, the notifying portion may be provided with: the conductive layer (contact detecting means) 31; a high-frequency detection portion (contact detecting means) 20 that is connected to the conductive layer 31 via an electric wire 19 that passes through the interior of the elongated insertion portion 2; and a speaker (recognizing means) 21 that is connected to the high-frequency detection portion 20.

When the electric scalpel 50 comes into contact with the conductive layer 31 of the sheet portion 3, which is exposed to the body cavity B from the incision site in the wall C, a high-frequency electric current output from the electric scalpel 50 is detected by the high-frequency detection portion 20 via the electric wire 19. The high-frequency detection portion 20 causes the speaker 21 to output a sound when the high-frequency electric current is detected. By doing so, it is possible to aurally notify the surgeon about penetration of the wall C, thus making him/her recognize penetration of the wall C. The surgeon may be made to recognize penetration of the wall C by using a visual means (for example, a warning display that is displayed on a display together with the image captured by the body cavity endoscope 40) instead of the sound.

In this embodiment, although a case in which the wall C that separates the lumen A and the body cavity B is treated has been described, the medical device 1 can be used to treat an arbitrary body wall that separates two cavities (for example, the diaphragm that separates the abdominal cavity and the chest cavity).

Although preferred embodiments have been described above, the present invention is not limited to the embodiments and modifications thereof. Additions, omissions, substitutions and other changes in the structure are possible without departing from the spirit of the present invention. The present invention is not limited by the foregoing description but is limited only by the scope of the appended claims.

### REFERENCE SIGNS LIST

1 medical device
2 elongated insertion portion
3 sheet portion
31 conductive layer
32 insulating layer
4 opening/closing mechanism
5 color layer (notifying portion)
51 reflector (notifying portion)
52 light-emitting body (notifying portion)
6, 61 attaching portion
61a suction port
13 suction pipe
14 suction device
17 earth line
20 high-frequency detection portion (notifying portion, contact detecting means)
21 speaker (notifying portion, recognizing means)
A lumen (cavity)
B body cavity (cavity)
C lumen wall (body wall)

What is claimed is:

1. A medical device comprising:
an insertion portion configured to be inserted into a body; and
a sheet provided at a distal end of the insertion portion, the sheet being configured to cover a body wall, the sheet comprising:
an attaching portion provided around a periphery of the sheet, the attaching portion being configured to directly contact with the body wall; and
a notifying portion for notifying a surgeon that an incision has been made through the body wall and towards a surface of the sheet.

2. A medical device according to claim 1, further comprising:
an opening-closing mechanism configured to cause the sheet to be deformed between an open form and an closed form;
wherein the open form being configured to increase a size of the sheet in a direction intersecting with a longitudinal direction of the insertion portion, and
the closed form being configured to decrease the size of the sheet in the direction intersecting with the longitudinal direction of the insertion portion as compared to the open form.

3. A medical device according to claim 1, wherein the notifying portion comprising a color layer disposed on the surface, a color of the color layer being substantially complementary to a color of the body wall.

4. A medical device according to claim 1, wherein the attaching portion being configured to protrude from the surface of the sheet in a longitudinal direction of the insertion portion.

5. A medical device according to claim 4, wherein the attaching portion comprising a biological adhesive configured to attach to the body wall.

6. A medical device according to claim 4, further comprising:
a suction device disposed at a proximal end of the insertion portion, the suction device configured to generate a suction force; and
a suction pipe configured to be extended along the insertion portion;
wherein the attaching portion comprising one or more suction ports configured to attach the attaching portion to the body wall by the suction force transmitted via the suction pipe.

7. A medical device according to claim 1, wherein the notifying portion comprising a reflector disposed on the surface, the reflector being configured to reflect light.

8. A medical device according to claim 1, wherein the notifying portion comprising a light-emitting body disposed on the surface, the light-emitting body being configured to emit light.

9. A medical device according to claim 1, wherein the notifying portion comprising:
a contact detecting unit provided on the sheet, the contact detecting unit being configured to detect contact with a treatment tool used to make an incision in the body wall with the sheet; and
a recognizing unit configured to notify the surgeon of contact of the treatment tool with the sheet.

10. A medical device according to claim 1, wherein the sheet comprising an insulating layer configured to possess electrical insulating properties.

11. A medical device according to claim 1, further comprising:
a ground line configured to be extended between the sheet and the proximal end of the insertion portion,
wherein the sheet comprising a conductive layer configured to possess conductivity,
wherein the ground line being configured to connect with the conductive layer.

12. A method for incising a neoplasm on a body wall, the body wall separating first and second cavities in a body, the method comprising:
inserting a first medical device comprising a sheet configured to cover a surface of the body wall into the first cavity;
inserting a second medical device configured to incise the neoplasm into the second cavity;
from a side of the first cavity, covering the surface of the body wall at a position of the neoplasm by the first medical device; and
from a side of the second cavity, incising the neoplasm by the second medical device.

13. The method according to claim 12, further comprising:
removing the second medical device from the second cavity;
inserting a third medical device configured to suture an opening made by incising the neoplasm into the second cavity; and
from the second cavity side, suturing the opening by the third medical device.

14. A medical system comprising:
a first medical device configured to incise an affected area at a body wall; and
a second medical device configured to cover an opposite surface of the body wall having the affected area, the second medical device comprising:
an insertion portion configured to be inserted into a body; and
a sheet provided at a distal end of the insertion port ion, the sheet being configured to cover the body wall, and the sheet comprising:
an attaching portion provided around a periphery of the sheet, the attaching portion being configured to directly contact with the body wall; and
a notifying portion for notifying a surgeon that an incision has been made by through the body wall and towards a surface of the sheet.

15. A medical system according to claim 14, wherein the second medical device further comprising:
an opening-closing mechanism configured to cause the sheet to be deformed between an open form and an closed form;
wherein the open form being configured to increase a size of the sheet in a direction intersecting with a longitudinal direction of the insertion portion, and
the closed form being configured to decrease the size of the sheet in the direction intersecting with the longitudinal direction of the insertion portion as compared to the open form.

16. A medical device according to claim 14, wherein the notifying portion comprising a color layer, a color of the color layer being substantially complementary to a color of the body wall.

17. A medical system according to claim 14, wherein the attaching portion being configured to protrude from the surface of the sheet in a longitudinal direction of the insertion portion.

18. A medical system according to claim 17, further comprising:
a suction device disposed at a proximal end of the insertion portion, the suction device configured to generate a suction force; and
a suction pipe configured to be extended along the insertion portion;
wherein the attaching portion comprising one or more suction ports configured to attach the attaching portion to the body wall by the suction force transmitted via the suction pipe.

19. A medical system according to claim 14, wherein the notifying portion comprising:
a contact detecting unit provided on the sheet, the contact detecting unit being configured to detect contact with the first medical device used to incise the affected area at the body wall with the sheet; and
a recognizing unit configured to notify the surgeon of contact of the first medical device with the sheet.

20. A medical system according to claim 14, the second medical device further comprising:
a ground line configured to be extended between the sheet and the proximal end of the insertion portion,
wherein the sheet comprising a conductive layer configured to possess conductivity,
wherein the ground line being configured to connect with the conductive layer.
